(19) United States
(12) Patent Application Publication
(10) Pub. No.: US 2016/0228067 A1
(43) Pub. Date: Aug. 11, 2016

(54) SYSTEM AND METHOD FOR INTELLIGENT MONITORING OF PATIENT VITAL SIGNS

(71) Applicant: TrulyWireless, LLC, Andover, MA (US)

(72) Inventor: Ravi Kuppuraj, Andover, MA (US)

(21) Appl. No.: 14/614,655

(22) Filed: Feb. 5, 2015

(51) Int. Cl. A61B 5/00; A61B 19/00; A61B 5/16; A61B 5/11; A61B 5/22

(52) U.S. Cl. CPC A61B 5/7264; A61B 5/0022; A61B 5/7275; A61B 5/7246; A61B 5/1112; A61B 5/222; A61B 5/4806; A61B 5/165; A61B 5/162; A61B 5/746; A61B 19/44; A61B 5/7475

(57) ABSTRACT

System and method for monitoring a patient is disclosed. The system and method provides a diagnosis of the patient based on a collective analysis of multiple sensor readings of the patient, environmental factor around the patient, and patient's physical behavioral patterns. The system and method also considers interaction between the patient and an authorized personnel to reach to the diagnosis, where one or more processors works together in a networked system environment.

### BACKGROUND

1. Field of the Invention

The present invention relates generally to a system and method for monitoring physiological data of a patient. More specifically, the present disclosure provides a system and method for detecting abnormalities of the patient, diagnosing the patient, and providing alerts and executable instruction therefrom.

2. Description of Related Art

With wireless communication becoming inexpensive and ubiquitous, a plethora of patient sensors that can transmit wirelessly are increasingly available. These sensors leverage advancements in miniaturization, wireless and battery technologies, and the IoT (Internet of Things) standards and infrastructure. Currently, wireless network access is readily available to the public and applicable in various sectors of the industry, and costs for the same are going down. This is also true for Cloud technology based processing and storage. However, there is a need for a connected system patient vital signs monitoring system that can provide a scalable, low cost, reliable, secure, HIPAA compliant vital signs analyses and making access to the vital signs and analyses to authorized personnel anytime and anywhere, and enabling them to improve accuracy and details of the clinical decisions for a patient by timely interventions and actions.

When examining signals generated from sensors, human errors do occur. Often times, a combination of a multiple readings from a plurality of sensors can lead to varying follow-up actions. A signal itself does not give an accurate follow-up instruction to be followed by a user. Current physiological systems for monitoring patient's vitals often are bulky in structure and not accommodating for timely and accurate delivery of alerts and instructions. The sensors signals being delivered are often a collection of multiple mono-functional indications from a multitude of various sensors.

As such, there is a need for a method to process multiple sensor signals in combination to create a deeper diagnosis of the patient condition, and to reach a more accurate follow-up. There also is a need for a system and method for analyzing/detecting/producing multi-parametric aggregation of data generated by a plurality of sensors, patient data acquired separately, along with other behavioral patterns by the patient that may be obtained/derived by one or more of the sensors in the system. A need also arises in improving a system for patient vital signs monitoring that is capable of providing scalable and more accurate executable instructions to the user. In addition, there yet is another need for accommodating environmental conditions in which the patient is located, in order to analyze a more suitable diagnosis of the sensor signals, enabling the user to make better clinical decisions.

There are also needs for timely alerts when there are either issues relating either the monitoring system/apparatus or patient needs human attention. In addition, there are needs for ubiquitous access to more detailed information and reports regarding the patient condition.

### SUMMARY

The subject matter of this application may involve, in some cases, interrelated products, alternative solutions to a particular problem, and/or a plurality of different uses of a single system or article.

In one aspect, a system for continuously monitoring a patient is provided. The system may comprise one or more processors in communication with each other via a network and a storage unit. A plurality of sensors, operatively coupled to the patient, may acquire a sensor data of the patient, where each of the plurality of sensors is in communication with the one or more processors. The system may further comprise a sensor data aggregation module, which may collect the sensor data from the plurality of sensors. The sensor data may be stored in the storage unit.

The system yet may comprise a multi-parametric analysis module to generate a physiological index by collectively analyzing at least one of a physiological index data, a relation between the physiological index data, and a tendency of the physiological index data over time. The physiological index may indicate diagnosis of the patient. The physiological index data may comprise the sensor data acquired from at least one of the plurality of sensors.

In another aspect, a method for continuously monitoring a patient is provided. The method may be operated by one or more processors in communication with each other via a network. The method may begin with acquiring a sensor data from the patient from the plurality of sensors operatively coupled to the patient. The one or more processors may aggregate the sensor data from the plurality of sensors and store in the storage unit. The method may continue with the one or more processors generating a physiological index by collectively analyzing at least one of a physiological index data, a relation between the physiological index data, and a tendency of the physiological index data over time. The physiological index may indicate diagnosis of the patient. The physiological index data may comprise the sensor data acquired from at least one of the plurality of sensors.

In yet another aspect, a non-transitory computer readable medium storing executable instructions which, when executed, cause one or more processors to perform the methods above for monitoring a patient is provided.

### BRIEF DESCRIPTION OF THE DRAWINGS

FIG. 1 provides an overview of the system for monitoring a patient.

FIG. 2 provides an embodiment of the system for monitoring a patient.

FIG. 3 provides an embodiment of the system for monitoring a patient for multiple patients.

FIG. 4 provides an exemplary embodiment of the primary computing device for monitoring a patient.

FIG. 5 provides an exemplary embodiment of the remote server for monitoring a patient.

FIG. 6 provides an exemplary embodiment of the clinical intelligence module.

FIG. 7 provides an exemplary embodiment of generating the physiological index.

FIG. 8 provides an exemplary flowchart describing the process of generating the physiological index.

### DETAILED DESCRIPTION

The detailed description set forth below in connection with the appended drawings is intended as a description of presently preferred embodiments of the invention and does not represent the only forms in which the present invention may be constructed and/or utilized. The description sets forth the functions and the sequence of steps for constructing and operating the invention in connection with the illustrated embodiments.

In referring to the description, specific details are set forth in order to provide a thorough understanding of the examples disclosed. In other instances, well-known methods, procedures, components and materials have not been described in detail as not to unnecessarily lengthen the present disclosure.

It should be understood that if an element or part is referred herein as being “on”, “against”, “in communication with”, “connected to”, or “coupled to” another element or part, then it can be directly on, against, in communication with, connected or coupled to the other element or part, or intervening elements or parts may be present. When used, term “and/or”, includes any and all combinations of one or more of the associated listed items, if so provided.

The terminology used herein is for the purpose of describing particular embodiments only and is not intended to be limiting. As used herein, the singular forms “a”, “an”, and “the”, are intended to include the plural forms as well, unless the context clearly indicates otherwise. It should be further understood that the terms “includes” and/or “including”, when used in the present specification, specify the presence of stated features, integers, steps, operations, elements, and/or components, but do not preclude the presence or addition of one or more other features, integers, steps, operations, elements, components, and/or groups thereof not explicitly stated.

Some embodiments of the present invention may be practiced on a computer system that includes, in general, one or a plurality of processors for processing information and instructions, RAM, for storing information and instructions, ROM, for storing static information and instructions, a data storage unit such as a magnetic or optical disk and disk drive for storing information and instructions, modules as software units executing on a processor, an optional user output device such as a display device (e.g., a monitor) for displaying information to the computer user, and an optional user input device.

As will be appreciated by those skilled in the art, the present examples may be embodied, at least in part, a computer program product embodied in any tangible medium of expression having computer-usable program code stored therein. For example, some embodiments described below with reference to flowchart illustrations and/or block diagrams of methods, apparatus (systems) and computer program products can be implemented by computer program instructions. The computer program instructions may be stored in computer-readable media that can direct a computer or other programmable data processing apparatus to function in a particular manner, such that the instructions stored in the computer-readable media constitute an article of manufacture including instructions and processes which implement the function/act/step specified in the flowchart and/or block diagram. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

In the following description, reference is made to the accompanying drawings which are illustrations of embodiments in which the disclosed invention may be practiced. It is to be understood, however, that those skilled in the art may develop other structural and functional modifications without departing from the novelty and scope of the instant disclosure.

Generally, the present invention concerns a system and method for monitoring a patient and providing examination, diagnosis, and treatment instruction for the patient. The system, with one or more processors employed in a networked environment, gathers sensor data from a plurality of sensors attached to the patient. The system may continuously monitor the patient for each of the plurality of sensors' corresponding physiological readings which is then accessible by an authorized personnel, such as doctors, nurses, and the like. The sensor data may be aggregated and analyzed to generate a physiological index which may indicate, among other things, the condition of the patient, overall health score of the patient, instructions, diagnosis, and examination of the patient. The authorized personnel may receive the physiological index via a client device to perform any actions of interventions to treat the patient or simply to monitor the patient's physical state.

In generating the physiological index, multiple physiological index data may be analyzed, in addition to the sensor data. Patient's movement, behavior and activity may be measured and received as a part of the physiological index data by the system to come to a more accurate physiological index. In addition, information unique per patient may also be included in generating the physiological index. Patient's medical history or patient's family medical history may be considered as physiological index data. Similarly, patient's current condition may be inputted by the patient self or by the authorized personnel to the system to improve the physiological index. Further, environmental condition specific to the patient's location may be considered to generate the physiological index.

Storage unit contemplated herein may be in the format including, but are not limiting to, XML, JSON, CSV, binary, over any connection type: serial, Ethernet, etc. over any protocol: UDP, TCP, and the like.

Computer or computing device contemplated herein may include, but are not limited to, virtual systems, Cloud/remote systems, desktop computers, laptop computers, tablet computers, handheld computers, smart phones and other cellular phones, and similar internet enabled mobile devices, digital cameras, a customized computing device configured to specifically carry out the methods contemplated in this disclosure, and the like.

Network contemplated herein may include, for example, one or more of the Internet, Wide Area Networks (WANs), Local Area Networks (LANs), analog or digital wired and wireless telephone networks (e.g., a PSTN, Integrated Services Digital Network (ISDN), a cellular network, and Digital Subscriber Line (xDSL)), radio, television, cable, satellite, and/or any other delivery or tunneling mechanism for carrying data. Network may include multiple networks or sub-networks, each of which may include, for example, a wired or wireless data pathway. The network may include a circuit-switched voice network, a packet-switched data network, or any other network able to carry electronic communications. Examples include, but are not limited to, Picture Transfer Protocol (PTP) over Internet Protocol (IP), IP over Bluetooth, IP over WiFi, and PTP over IP networks (PTP/IP).

Sensors contemplated herein may monitor patient's vitals, which may include, but are not limited to, Heart Rate, Blood Pressure(s), body weight, concentration of one or more metabolite(s) in the blood, concentration of one or more gas(es) in the blood, temperature, Asystole, Respiration, electrocardiogram. patient vital signs, but not limited to: Respiration, patient activity from accelerometer(s), patient activity from gyroscope(s), ECG beat detection and classification, ECG rhythm classification, ECG interpretation, ECG-ST segment analysis, ECG-QT measurement, Cardiac Output, Heart Rate Variability, Temperature(s), Blood gas (including oxygen) concentration/saturation, metabolite concentration in body fluids.

Sensor, may include, but are not limited to, a sensor circuit detecting a ECG signal(s), a sensor circuit detecting a respiration rate signal indicative of the breathing of the patient and a sensor circuit detecting the movement and/or posture of the patient, such as an accelerometer, 3-axis accelerometer, altimeter, gyroscope, and the like.

The data produce by the sensor may include any type of data, by way of non-limiting examples: a static image derived from but not limited to the following imaging techniques or modalities: optical/photographic, infra-red, magnetic resonance imaging (MRI), ultra-sound imaging, x-ray, computerized tomography (CT), and positron emission tomography (PET). Dynamic images/video derived from but not limited to the following imaging optical/photographic, infra-red, magnetic resonance imaging (MRI), ultra-sound imaging, x-ray, computerized tomography (CT), and positron emission tomography (PET).

Camera contemplated herein may include, but are not limited to, DSLR, non-SLR digital cameras (e.g., but not limited to, compact digicams and SLR-like bridge digital cameras (also known as advanced digital cameras), and SLR-like interchangeable lens digital cameras), as well as video recorders (e.g., but not limited to, camcorders, analog cameras and IP cameras, and the like; a device that can provide a video feed of any duration, such as a DVR; a portable computing device having a camera, such as a tablet computer, laptop computer); and the like.

The system for monitoring a patient is provided. The system may comprise a plurality of sensors and a computerized system with one or more processors, and a storage unit accessible by the one or more processors via a network. The system may comprise one or more computers or computerized elements in communication working together to carry out the different functions of the system. The invention contemplated herein further may comprise non-transitory computer readable media configured to instruct a computer or computers to carry out the steps and functions of the system and method, as described herein. In some embodiments, the communication among the one or more computer or the one or more processors alike, may support a plurality of encryption/decryption methods and mechanisms of various types of data.

The computerized user interface may be comprised of one or more computing devices in networked communication with each other. The computer or computers of the computerized user interface contemplated herein may comprise a memory, processor, and input/output system. In some embodiments, the computer may further comprise a networked connection and/or a display. These computerized elements may work together within a network to provide functionality to the computerized user interface.

The computerized user interface may be any computerized interface capable of allowing a user to input data and receive a feedback. Data input may include patient information, medical history, patient condition, and the like. The computerized user interface may further provide outputs including instructions received by the physiological index and display on a screen, audible, or the like.

In one embodiment, the system may comprise a plurality of sensors attached to the patient in order to read vital signs or physiological data of the patient, which hereinafter referred to as sensor data. The plurality of sensors may transmit sensor data to the one or more processors via a network. In some embodiment, the sensor data may be transmitted in a wired or wireless network. In some embodiments, the plurality of sensors may comprise a transmitter integrated therein to transmit the sensor data to the one or more processors, where the sensor data is received by the one or more processors by a receiver in communication with the one or more processors.

The system may employ a multiple computing devices enabling patient-to-authorized personnel interaction and communication. The multiple computing devices each may be in communication with and operated by at least one of the one or more processors.

In one embodiment, the one or more processors may comprise a remote processor and a primary processor to carry out the method described herein.

In another embodiment, the system may comprise a primary computing device. The primary computing device may be accessible and placed within the vicinity of the patient. The primary computing device may be in communication with or operated by at least one of the one or more processors. The primary computing device may comprise a primary analysis module which may monitor and analyze an operational status of the primary computing device. The primary analysis module also may indicate the operational status and a part of the sensor data. The primary computing device may further comprise a display or a primary status indicator to display basic abnormalities detected by the sensor data. In some embodiment, the primary computing device may detect the operational status of the primary computing device, such as battery status, network status between the primary computing device and the one or more processors, and the like.

In a further embodiment, the system may comprise an alarm module. The alarm module may be in communication with the primary computing device which issues an alarm when the operational status indicates malfunction. By way of non-limiting example, the alarm module may alert the patient to charge the battery or a power source of the primary computing device when it is near depleted. Similarly, the alarm module may alert the patient to check or repair the network status of the system and communication link between the one or more processors and the primary computing device. As well known to those with ordinary skill in the art, the alarm module may employ various alarming devices, such as audible, textual, visual alarms, and the like.

In a yet another embodiment, the alarm module may issue the alarm when critical sensor data is abnormal. The abnormalities may be detected by the primary computing device by comparing the sensor data to an expected value. Each of the plurality of sensors is assigned with an expected value accessible from the storage unit.

The system may comprise a remote processor to monitor, examine, and diagnose the patient. Multiple modules may be in communication with the remote processor to carry out the functions described herein. Similarly, components of the system, such as the one or more processors, may be in remote communication with one another and employed in more than one computing devices to carry out the functions described herein. The storage unit may be accessible by the remote processor to store and access data received by the remote processor. The utilization of the remote processor enables the patient and the authorized personnel to be in contact regardless of their locations. The remote processor is commonly placed in a cloud networking environment to be accessible by one or more processors and computing devices.

In one embodiment, the one or more processor may provide a multi-parametric analysis of the sensor data. The system may comprise a multi-parametric analysis module. The multi-parametric analysis module may generate a physiological index by collectively analyzing a physiological index data, which may comprise the sensor data obtained from the plurality of sensors. The physiological index data may be analyzed along with a relation between each of the physiological index data and a tendency of the physiological index data over time. The sensor data may be recorded in the storage unit over a duration of the patient monitoring. The multi-parametric analysis module may compare the physiological index data, the relation, and the tendency obtained from the patient with a model guideline and protocol to generate the physiological index. The physiological index may indicate, among other things, the condition of the patient, overall health score of the patient, instructions, diagnosis, and examination of the patient.

Conventional gathering of sensor data is analyzed without correlating two or more of the plurality of sensor reading. It analyzes the plurality of readings from the plurality of sensors, then compares each of the plurality of readings to the corresponding threshold level. The multi-parametric analysis module may receive the plurality of sensor data aggregated by a sensor data aggregation module. The multi-parametric analysis module then may analyze the plurality of sensor data based on not only the individual sensor data, but also the correlations among the plurality of sensor data. This feature may prevent human errors and mis-diagnosis, most importantly an accurate instruction of intervention by the authorized personnel.

By way of non-limiting example, minor abnormalities form the plurality of sensors, when individually analyzed may not trigger the physiological index to indicate a warning. On the other hand, when minor abnormalities from the plurality of sensors are collectively analyzed with the relation among each of the sensor data and the tendency, the resulting physiological index may indicate a patient condition that requires the authorized personnel's intervention to improve the patient condition. In another example, when a lower than threshold sensor data from one of the plurality of sensors persists as it is over time, in other words the sensor data analyzed in light of its tendency, the physiological index may require an intervention.

The system may further diagnose and monitor the physical activity of the patient and any behavioral pattern obtained therein. The physiological index data may comprise data from monitoring the patient in such way, thereby contributing to generate the physiological index.

In one embodiment, the system for monitoring a patient may comprise a motion monitoring unit. The motion monitoring unit may sense physical movement or activity of the patient, hereinafter referred to as “motion behavior data”. The motion monitoring unit may be an accelerometer, 3-axis accelerometer, and the like. The motion monitoring unit may be in communication with the one or more processors, and positioned to track the motion behavior data of the patient. The motion behavior data of the patient as tracked may be stored and its trend may be analyzed. In one embodiment, the motion behavior data may comprise an exercise activity of the patient. The exercise activity, such as running, workout, or climbing steps may be monitored and recorded. By way of non-limiting example, the exercise activity may be logged daily to monitor frequency and duration of the patient's physical activity, as these may contribute to the change, improvement, and/or worsening of the patient's condition.

In another embodiment, the motion behavior data may comprise a sleep activity of the patient. The motion monitoring unit may acquire the duration of sleep and quality of sleep by monitoring the movement of the patient while asleep.

In yet another embodiment, the motion behavior data may comprise a stress level. The motion monitoring unit may indicate that the patient had an excessive physical activity or an insufficient duration of sleep which may lead to higher stress level.

In a further embodiment, the motion monitoring unit may identify calories spent by the patient by observing the patient's physical activity.

In a further embodiment, the motion monitoring unit may detect the patient's movement upon the system requesting the patient for a response. A response time of the patient may be acquired by measuring the time it takes for the patient to respond to the system requesting a movement. By way of non-limiting example, the system may prompt the user to move his/her finger, depending on the response time of the patient moving the finger, the motion behavior data may affect the physiological index.

By way of non-limiting example, the physiological index may be based on the motion behavior data derived from the accelerometer (i.e. sleep quality, activity level, calories burnt, etc.). In one embodiment, the sleep quality of the patient may be identified by employing the accelerometer attached to the patient. The accelerometer may record the duration of sleep the patient acquired, by observing the idle period of the accelerometer. In another example, level of the exercise activity may be identified by measuring the amount of movement the patient has achieved during the day, thereby identifying whether the patient had enough exercise for the day.

The following shows a non-limiting example of collectively analyzing the motion behavior data and the sensor data to come to the physiological index. The return to normal heart rate/ECG and breathing after exercise is called recovery time. The quicker/shorter this time is, the fitter a person is. The recovery time also may depend on the duration and intensity of the exercise activity. When a healthy person exercises, the heart rate, breathing rate and lactic acid levels rise much less than they do in a person who has health issues. The time which it takes for HR and breathing to return to normal is called the recovery time. The fitter you are, the shorter your recovery time is. Thus, the recovery time is the time it takes to replenish the oxygen consumed by the body. If you are fit you make less lactic acid, as such there is less to get rid of. An efficient heart and lungs provide the oxygen much more quickly. The accelerometer can characterize the duration and intensity of the exercise activity. The recovery time or the presence of any other abnormalities after such exercise activity could indicate a negative physiological index.

By way of another non-limiting example, when blood pressure level acquired from the sensor data is over 140/90, alone it is classified as Hypertension. Thus, based on the sensor data itself, the physiological index may require a treatment accordingly. On the other hand, even when blood pressure level is over 140/90, it may not require a physiological index of the same, when the physiological index data of a patient shows that the ECG and Respiration are under normal limits (sensor data), in combination with a period of exercise activity (motion behavior data).

In another example, when BP, heart rate, and respiration is a bit over the normal thresholds of (120/80, 80 bpm, and 18 breaths per minute, respectively), the individual analysis of theses sensor data may not alarm the authorized personnel. However, when analyzed in combination and with poor sleep quality, weight gain, etc. these close-to-threshold readings could be symptom of congestive heart failure (CHF).

The physiological index may also be affected by information specific to the patient. Every individual patients requires different standards and adjustment to accurately diagnose the patient. The system may further comprise an identification module. The identification module may acquire data specific to the patient. The identification module may be in communication with the one or more processors.

In one embodiment, the identification module may acquire medical history of the patient. The medical history of the patient may be stored in the storage unit accessible by the one or more processors. Medical history of the patient may indicate prior ailments that the patient had in the past, subsequently affecting the physiological index of the patient. Medical history may also comprise information about test data and results of the patient, as the patient undergoes any test or treatment such data may constitute the medical history.

In another embodiment, the identification module may acquire patient condition at the current state. The patient condition may be received by the identification module via the computerized user interface from the patient or the authorized personnel. By way of non-limiting example, the input of the patient condition from the authorized personnel may affect the physiological index. The authorized personnel may observe the patient or examine the patient for symptoms or signs that is not available to be accurately acquired by the sensors. The authorized personal may make a note that the patient is “slurring speech”, such symptom may affect the physiological index. In this example, the patient condition “slurring speech” may be an indication that points to an ailment or treatment that is indexed and accessible in the storage unit. “slurring of speech” is a common symptom for serious life-threatening conditions, such as stroke or traumatic brain injury, as such the physiological index may require an intervention, treatment, or action from the authorized personnel. In a case where the sensor data do not indicate any abnormalities, the note of “slurring of speech” by the authorized personnel may accurately assign the physiological index that instructs the doctor to take action. In addition, similarly, pain level, loss of sensation/numbness, fatigue and the like may be an input of the patient condition.

In yet another embodiment, the identification module may verify the patient's identity by receiving patient identification (for example, name, social security number, date of birth, etc.) from the patient via the computerized user interface.

In a further embodiment, the identification module may verify the patient's identity by observing the patient's biometrics and other individually unique biological and/or physiological signature of the patient. For example, ECG signals, breathing patterns, finger prints, and the like are unique biological and physiological signature of individuals.

In a further embodiment, the identification module may detect a patient interaction with the system when the system requests the patient for an action. A response time of the patient may be acquired by measuring the time it takes for the patient to perform the action requested by the system. The patient interaction also may be measured by the accuracy in which the patient performs the action requested by the system. By way of non-limiting example, the system may prompt the user with a question, such as “What is your address?”, depending on the response time of the patient answering the question and the accuracy of the answer, the patient interaction may affect the physiological index, either negatively or positively.

In a further embodiment, the identification module may detect the patient interaction with the system by observing behavior of the patient interacting with the system. The system may be in communication with a computing device of the patient. In this embodiment, the system may monitor the patient's behavior with the computing device. Behaviors such as frequency in use, time of use, type of web sites visited, information inputted and outputted with the computing device may be monitored by the identification module. By way of non-limiting example, for a patient who is being monitored, the impending onset of ailment can be determined by any accompanying behavior or change in behavior of the patient as determined and derived by their use of social media, such as lack of use, or the use of very specific language and terms in “posts” by the patient, or visits and/or searches to such “posts” or Blogs, website, and the like.

In a further embodiment, the identification module may be in communication with a medical protocol database via the network. The medical protocol database may provide medical protocols or guidelines for treatment and diagnosis of the patient. For example, Sepsis protocols and Early Warning Scores may be provided and take part in generating the physiological index. Such databases may be available via the internet or patient's hospital database.

By way of non-limiting example, for a patient who is being monitored for CHF, the impending onset of CHF can be determined by the combined abnormalities of rapid Respiration Rate of greater than 20 breaths per minute (shortness of breath, hyperventilation), palpitations as detected by ECG/HR, weight gain, along with stress as either derived by the system (sleep quality, activity), along with pain/fatigue as indicated by either the patient or the authorized personnel.

The system may further analyze environmental factor around the patient. The system may identify location of the patient and may consider data specific to the patient's location. The physiological index data may further comprise such data, thereby contributing to the physiological index.

The system may further comprise a clinical intelligence module. The clinical intelligence module may be in communication with the one or more processor. Further, the clinical intelligence module may be in communication with the internet to gather data specific to the patient's location.

In one embodiment, the location of the patient may be identified by the location of the patient's hospital or home, where the patient is being monitored. In another embodiment, the location may be identified by a location tracking unit. The location tracking unit may be in communication with the one or more processor. The location tracking unit may also be operatively placed to detect and monitor the patient's location. The location tracking unit may include, but not limited to, a GPS tracking unit and the like. Similarly, the location tracking unit may be positioned within a mobile computing device, such as a cell phone and the like. As such, the location tracking unit within the mobile computing device may be accessible by the one or more processor.

The clinical intelligence module may generate a clinical intelligence data that is the data specific to the patient's location. The physiological index data may further comprise the clinical intelligence data, thereby contributing to generating the physiological index. The multi-parametric analysis module may correlate the sensor data with the clinical intelligence data to come to a more accurate physiological index.

In one embodiment, the clinical intelligence data may comprise an environmental condition, such as weather condition, air pollution data, and the like. In another embodiment, the clinical intelligence data may comprise a seasonal ailment trend, such as a flu season and the like. In yet another embodiment, the clinical intelligence data may comprise an ailment outbreak, such as a localized measles outbreak. By way of non-limiting example, if the patient's body temperature is slightly lower than normal and the patient's environment was in outdoor area during a winter storm, the physiological index may indicate that the drop in patient's body temperature is normal. On the other hand, if the patient's body temperature appears slightly lower than normal during a hot summer day, the physiological index may instruct the authorized personnel to intervene.

The system for monitoring the patient may further comprise a client device. The client device may be accessible to the authorized personnel to receive the physiological index. The client device may be a computing device. The one or more processor may be in communication with the client device and may transmit the physiological index to the client device. The client device may provide an interface between the system and the authorized personnel. In some embodiments, the client device may comprise a computerized user interface to interact with the patient's device or to interact with the system itself. In one embodiment, the physiological index may be accessible to the client device in multiple types of representation, which may include, but not limited to, audio, video, numeric, color-coded, textual, and the like.

As described above and herein, the physiological index of the present invention may be improved by factoring in a variety of data not limiting to the plurality of sensors attached to the patient, at the least. The physiological index may be generated based on the physiological index data which may comprise the sensor data, the motion behavior data, patient's medical history, the patient condition, the patient interaction, and the clinical intelligence data, as described above. In most embodiment, any combination of the physiological index data may be collectively analyzed to accurately diagnose the patient, and to prevent any false positives. The multi-parametric analysis module may collectively analyze the physiological index data, the relation between the physiological index data, and the tendency of the physiological index data over time.

The system may further comprise a report generator. The report generator may be in communication with the one or more processor. The report generator may provide a report of the patient being monitored to the client device or the computerized user interface via the network. The report may include the sensor data, the physiological index, and any combination of the data availed by the system provided herein.

A method for monitoring a patient is provided. The method may be employed by the system provided above, and may comprise the process and interaction among the components of the system provided above to carry out different functions described herein. In one embodiment, the method may begin by gathering the sensor data from the plurality of sensors. The sensor data from the plurality of sensors may be aggregated and collectively analyzed, resulting in the physiological index. The physiological index may be generated in light of the sensor data, the relation between the sensor data, and the tendency of the sensor data over time.

In another embodiment, the method may comprise monitoring of the operational status of the system or the primary computing device. Further, the one or more processor or the primary computing device may further issue an alarm. The alarm may be issued based on the operational status of the system. When there is a malfunction in operation of the system or the primary computing device, the alarm may be issued. The method, thus, may further comprise comparing the operational status against an expected status to issue the alarm.

In yet another embodiment, the method may comprise acquiring various types of the physiological index data. As described above, in this embodiment, the method may comprise acquiring at least one of the motion behavior data, the sensor data, the medical history, the patient condition, the patient interaction, and the clinical intelligence data.

In a further embodiment, the method may comprise transmitting the physiological index to the client device via the network. Similarly, generating and transmitting the report may be a part of the method.

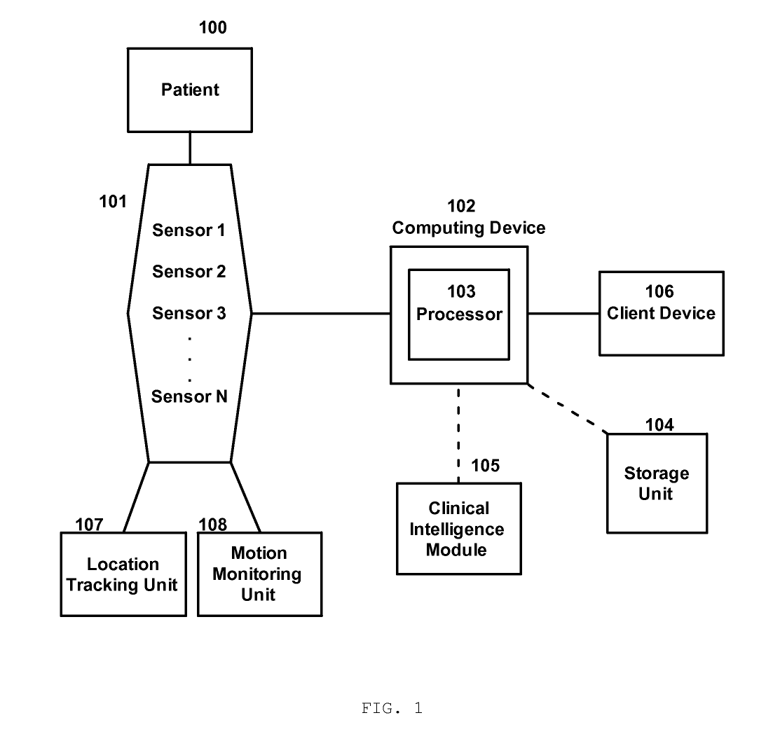

Turning now to FIG. 1, an overview of the system for monitoring a patient is illustrated. In this exemplary embodiment, the system employs one processor 103 within a computing device 102, where the processor 103 is remotely in communication with a storage unit 104. The data received by the system or other data may be stored within the storage unit 104 and accessible to the components and devices of the system. The storage unit 104 may hold computer program instructions and/or modules to carry out the functions disclosed above. The plurality of sensors 101 is coupled to the patient 100 to read the sensor data. In addition, location tracking unit 107 and motion monitoring unit 108 track location and movement of the patient, respectively. The sensor data gathered by the plurality of sensors 101, the patient location tracked by the location tracking unit 107, and the motion behavior data tracked by the motion monitoring unit 108 are accessible by the processor 103. The clinical intelligence module 105 is in communication with the computing device 102 where it generates the clinical intelligence data. The physiological index or any data availed by the processor 103 are accessible to the authorized personnel via the client device 106.

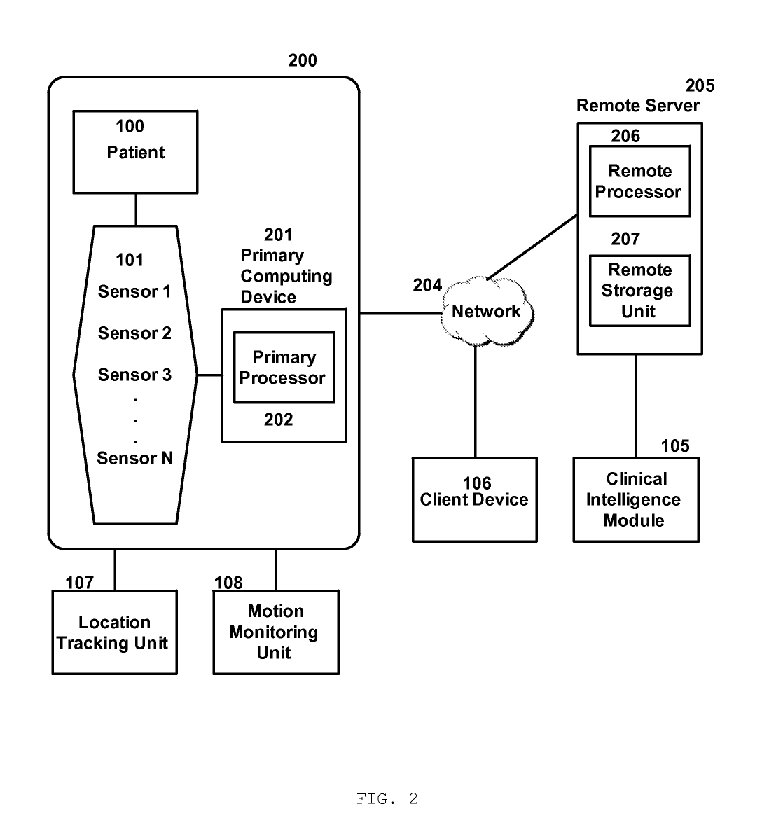

FIG. 2 describes an embodiment of the system for monitoring patient. In this exemplary embodiment, the system utilizes two processors communicating via a network to carry out the functions of the system. In the patient's vicinity 200, the system provides the plurality of sensors 101 attached to the patient 100. The sensor data from the plurality of sensors may be processed at the primary processor 202 and/or the remote server 205. In the patient's vicinity 200, the patient may have an access to the operational status of the system via the primary computing device 201. The primary computing device 201 is in communication with the client device 106 through the network 204, which enables the patient interaction with the authorized personnel and the system itself. The location tracking unit 107 and the motion monitoring unit 108 are operationally coupled to the primary computing device 201 for monitoring the patient 100. The remote server 205 comprises the remote processor 206 and the remote storage unit 207. Some or all of the modules may be operational at the remote server 205. The clinical intelligence module 105 is further in communication with the remote server which gathers the clinical intelligence data, then it is stored in the remote storage unit 207.

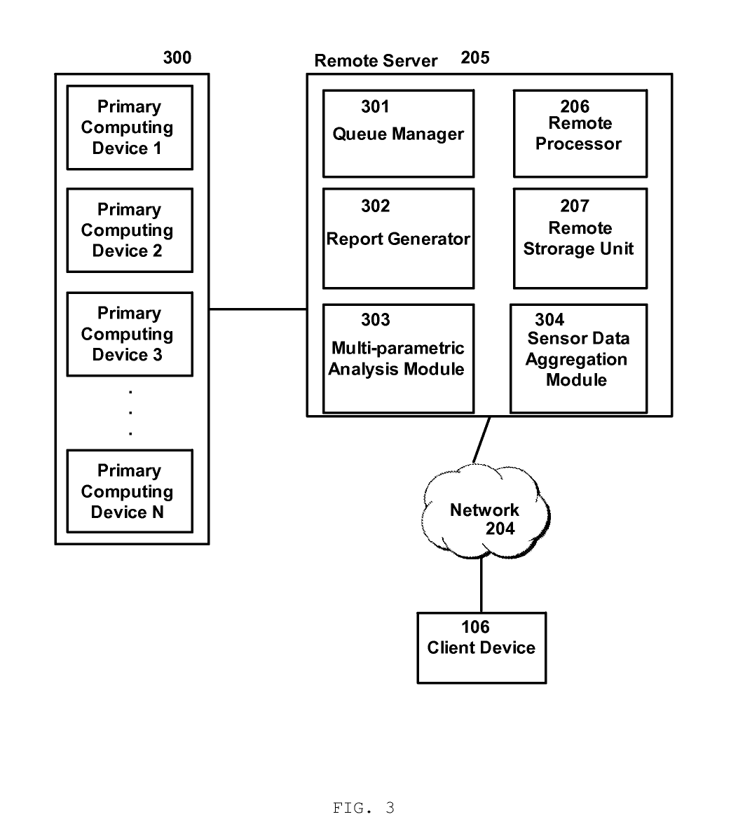

FIG. 3 illustrated an exemplary embodiment of the system for monitoring a multiple patients. A plurality of primary computing devices 300 are in communication with the remote server 205. Each of the plurality of primary computing devices is assigned to each of the multiple patients for observing the multiple patients. The remote server 205 comprises a queue manager 301 which queues the sensor data or other type of physiological index data generated from each of the plurality of primary computing devices 300. The remote server 205 further comprises a remote processor 206 and a remote storage unit 207 for remotely processing data gathered from the multiple patients. The sensor data aggregation module 304 aggregates sensor data from each of the plurality of primary computing devices 300. Once aggregated, the multi-parametric analysis module 303 generates the physiological index. The report may be generated by the report generator 302. The physiological index, the report, and the system may be accessible by the authorized personnel via the client device 106 in communication with the remote server 205 through the network 204.

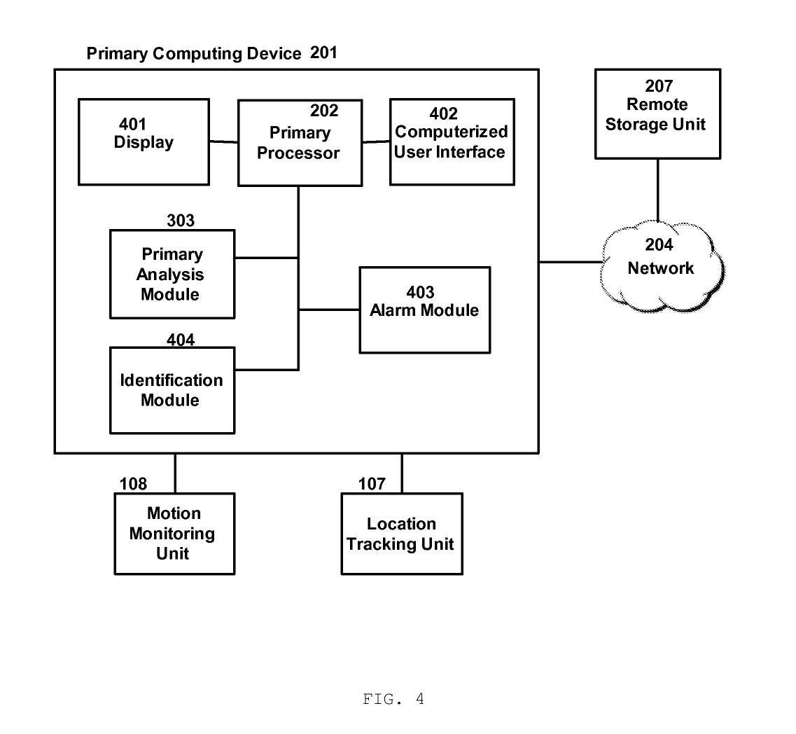

FIG. 4 describes an exemplary embodiment of the primary computing device for monitoring a patient. The primary computing device 201 comprises a primary processor 202. In this embodiment, the primary computing device 201 provides a display 401 to indicate any alarms or display data available by the system. A computerized user interface 402 allows the patient to interact with the primary computing device 201. Via the network 204, the primary computing device 201 can communicate to the authorized personnel and have access to the remote storage unit 207. The primary analysis module 303 monitors the operational status of the system components and the alarm module 403 issues an alarm when the system components malfunctions. In this embodiment, the indication of malfunctions can be shown on the display 401. The primary computing device 201 further comprise an identification module 404 which identifies the patient by interacting with the patient through the computerized user interface 402. The motion monitoring unit 108 and the location tracking unit 107 further observes the patient and are in communication with the primary computing device 201.

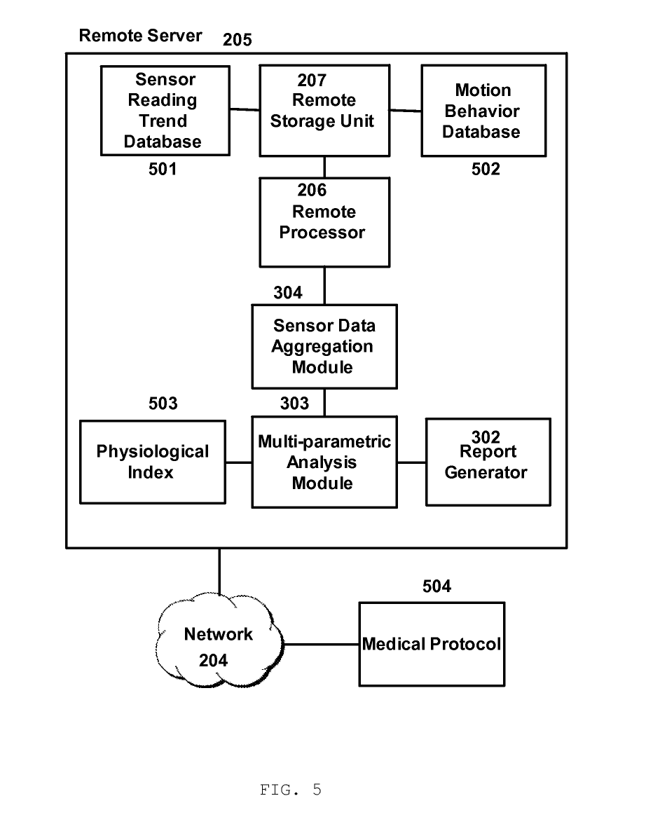

FIG. 5 illustrates an exemplary embodiment of the remote server for monitoring a patient. In FIG. 5, the remote server 205 carries out the computer program instructions to analyze the physiological index data. The remote server storage unit 207 holds data related to the tendency of the sensor data in a sensor reading trend database 501. In this database, tendency, changes in the sensor data or physiological index data are identified. The remote storage unit 207 further is in communication with the motion behavior database 502 storing the motion behavior data gathered by the motion monitoring unit. The remote processor 206 communicates with the sensor data aggregation module 304 and the multi-parametric analysis module 303 to analyze the sensor data and the physiological index data gathered from monitoring the patient, these data is accessible from monitoring the patient via the network 204. In addition, the physiological index data may further comprise a medical protocol 504 data. The medical protocol 504 provides guidelines for treatment and diagnosis of the patient. For example, current and emerging protocols (e.g. Sepsis protocols, Early Warning Scores) may be available from the medical protocol 504. In combination with the sensor data and the physiological index data, the medical protocol may provide a suitable symptom, diagnosis, or treatment for the monitored patient condition. The report generator 302 generates a report that contains physiological index 503. The physiological index 503 may be accessible to the users of the system through the network 204.

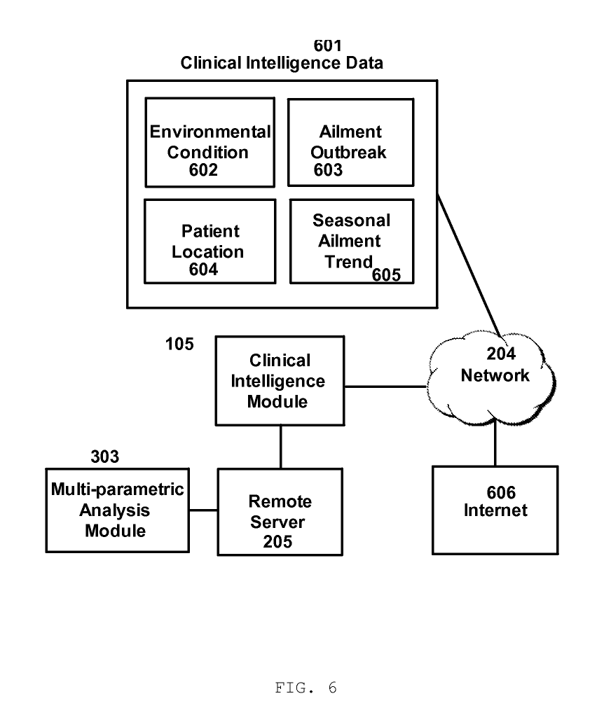

FIG. 6 discloses an exemplary embodiment of the clinical intelligence module in the system for monitoring a patient. The clinical intelligence module 105 has access to the internet 606 for gathering clinical intelligence data 601 specific to the patient's location, such as the environmental condition 602, the patient location 604, the ailment outbreak 603, and the seasonal ailment trend 605. The clinical intelligence module 105 may further gather data through the network 204 from other components of the system. The clinical intelligence module 105 is operated by the remote server 205. Among many functions of the system, the multi-parametric analysis module 303 receives the clinical intelligence data 601 to arrive at the physiological index by analyzing the clinical intelligence data 601 collectively with the sensor data.

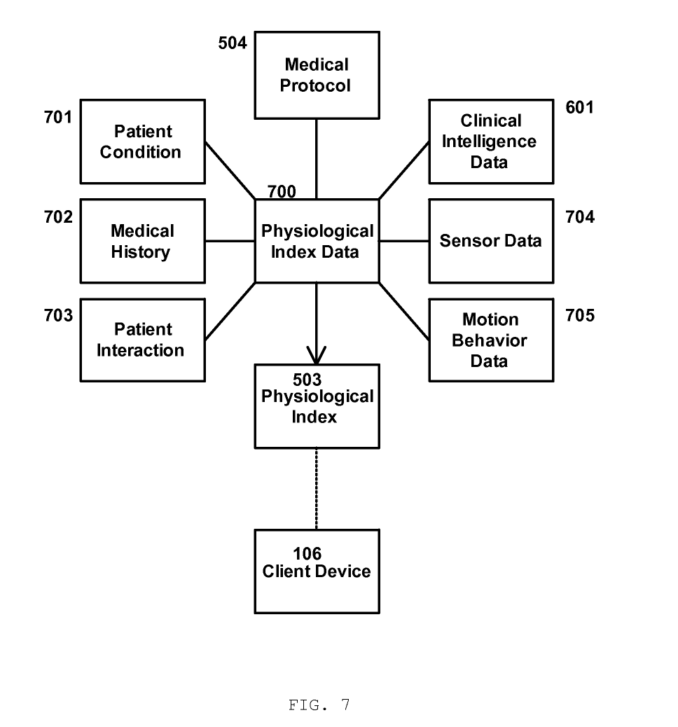

FIG. 7 shows a schematic diagram of generating the physiological index. In this exemplary schematic diagram, the physiological index data 700 is gathered from the identification module for the patient condition 701, the medical history 702, and the patient interaction 703. Further, the physiological index data 700 comprise the clinical intelligence data 601 from the clinical intelligence module, the sensor data 704 from the plurality of sensors, the motion behavior data from the motion monitoring unit. The physiological index 503 is generated by the multi-parametric analysis module based on the physiological index data 700, the relation between the physiological index data, and the tendency of the physiological index data over a period of time monitoring the patient. The abnormalities detected from comprehensively analyzing the physiological index data, the relation, and the tendency, may indicate a symptom, guideline, and diagnosis of the patient. In some embodiment, the medical protocol 504 may further be in communication with the multi-parametric analysis module, providing clinical/medical guidelines for a most up to date diagnosis of the observed abnormalities. The physiological index 503 is linked to the client device 106 for the authorized personnel's access to it.

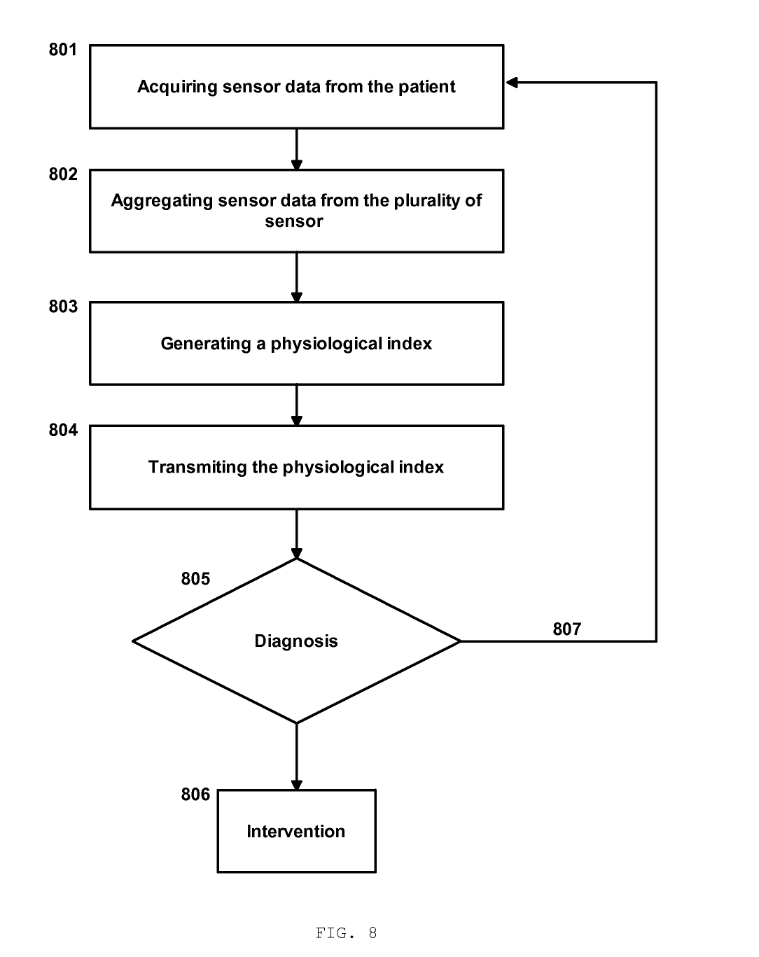

FIG. 8 describes an exemplary flowchart showing the process of generating the physiological index. The process begins with the plurality of sensors acquiring sensor data from the patient 801, then the sensor data aggregation module aggregating sensor data from the plurality of sensors 802. At 803, the physiological index is generated by collectively analyzing the sensor data, the relation between the sensor data, and the tendency of the sensor data over time. Once the physiological index is ready, it is transmitted to the client device 804. If the diagnosis indicated by the physiological index 805 is positive and requires no further action, the process repeats continuously 807. If the diagnosis 805 is negative, such that it requires the authorized personnel to intervene and treat the patient, the intervention 806 is presented to the client device.

While several variations of the present invention have been illustrated by way of example in preferred or particular embodiments, it is apparent that further embodiments could be developed within the spirit and scope of the present invention, or the inventive concept thereof. However, it is to be expressly understood that such modifications and adaptations are within the spirit and scope of the present invention, and are inclusive, but not limited to the following appended claims as set forth.

Those skilled in the art will readily observe that numerous modifications, applications and alterations of the device and method may be made while retaining the teachings of the present invention.

1. A system for continuously monitoring a patient, comprising:
one or more processors in communication with each other via a network, the one or more processors in communication with a storage unit;
a plurality of sensors operatively coupled to the patient, each of the plurality of sensors acquiring a sensor data of the patient, wherein each of the plurality of sensors is in communication with the one or more processors;
a sensor data aggregation module, in communication with the one or more processor, collecting the sensor data from the plurality of sensors, wherein the sensor data is stored in the storage unit;
a client device, in communication with the one or more processors, monitoring a behavior of the patient resulting from a response of the patient when an action is requested by the client device, wherein the client device acquires the behavior comprising a response time and a response accuracy, the response time indicating the time it takes for the patient to perform the requested action, and the response accuracy indicating the accuracy in which the patient performs the requested action; and
a multi-parametric analysis module, in communication with the one or more processors, configured to:
collectively analyze a plurality of physiological index data and a correlation between the plurality of physiological index data;
compare the correlation to a medical protocol stored in the storage unit; and
generate a physiological index based on the comparison, wherein the physiological index indicates diagnosis of the patient, the physiological index data comprising the sensor data acquired from at least one of the plurality of sensors, and the behavior.

2. The system of claim 1 further comprising:
a motion monitoring unit, in communication with the one or more processors, operatively positioned to monitor a movement of the patient, wherein the movement is requested by the client device as the action, the response time indicating the time it takes for the patient to perform the requested movement, and the response accuracy indicating the accuracy in which the patient performs the requested movement.

3. The system of claim 2 wherein the movement is selected from the group consisting of:
an exercise activity;
a sleep activity; and
an activity indicative of a stress level.

4. The system of claim 1 further comprising:
a primary analysis module, in communication with the one or more processors, the primary analysis module monitoring an operational status of the system; and
an alarm module, in communication with the primary analysis module, wherein the alarm module issues an alarm when the operational status indicates a malfunction.

5. The system of claim 1 further comprising an identification module, in communication with the one or more processors, the physiological index data further comprising data received by the identification module, wherein the identification module is configured to monitor
a behavior of the patient interacting with a web browser provided by the client device, wherein the behavior comprises at least one of a type of information accessed by the web browser, a frequency in use, a time of use, and a duration of use.

6. The system of claim 1 further comprising a clinical intelligence module, in communication with the one or more processors and the internet, the clinical intelligence module generating a clinical intelligence data, specific to the location of the patient, from the internet, wherein the physiological index data further comprises the clinical intelligence data, the clinical intelligence data being selected from the group consisting of:
an environmental condition;
a seasonal ailment trend; and
an ailment outbreak.

7. The system of claim 1 further comprising a location tracking unit, in communication with the one or more processors, operatively positioned to detect a patient location, wherein the patient location is stored in the storage unit.

8. The system of claim 1 wherein the physiological index is transmitted to the client device, wherein the client device provides a computerized interface between the system and an authorized personnel.

9. The system of claim 1 further comprising a report generator, in communication with the one or more processors, the report generator providing a report based on the sensor data and the physiological index data.

10. The system of claim 1 wherein the one or more processors comprises a remote processor.

11. A method for continuously monitoring a patient, the method operated by one or more processors, wherein the one or more processors is in communication with each other via a network, the one or more processors in communication with a storage unit, comprising the steps of:
acquiring a sensor data from the patient by a plurality of sensors, the plurality of sensors operatively coupled to the patient, wherein each of the plurality of sensors is in communication with the one or more processors;
aggregating the sensor data from the plurality of sensors, by the one or more processors, wherein the sensor data is stored in the storage unit;
monitoring a behavior of the patient, using a client device in communication with the one or more processors, resulting from a response of the patient when an action is requested by the client device, wherein the behavior comprises a response time and a response accuracy, the response time indicating the time it takes for the patient to perform the requested action, and the response accuracy indicating the accuracy in which the patient performs the requested action;
collectively analyzing, by the one or more processors, a plurality of physiological index data and a correlation between the plurality of physiological index data;
comparing, by the one or more processors, the correlation to a medical protocol stored in the storage unit; and
generating, by the one or more processors, a physiological index based on the comparison, wherein the physiological index indicates diagnosis of the patient, the physiological index data comprising the sensor data acquired from at least one of the plurality of sensors, and the behavior.

12. The method of claim 11 further comprising the step of monitoring a movement of the patient, using a motion monitoring unit in communication with the client device, wherein the movement is requested by the client device as the action, the response time indicating the time it takes for the patient to perform the requested movement, and the response accuracy indicating the accuracy in which the patient performs the requested movement.

13. The method of claim 12 wherein the movement is selected from the group consisting of:
an exercise activity;
a sleep activity; and
an activity indicative of a stress level.

14. The method of claim 11 further comprising the steps of:
monitoring an operational status of the system, by the one or more processors; and
issuing an alarm, by the one or more processors, when the operational status of the system indicates a malfunction.

15. The method of claim 11 wherein the physiological index data further comprises:
a behavior of the patient interacting with a web browser provided by the client device, wherein the behavior comprises at least one of a type of information accessed by the web browser, a frequency in use, a time of use, and a duration of use.

16. The method of claim 11 further comprising the steps of:
identifying a patient location with a location tracking unit, the location tracking unit in communication with the one or more processors;
gathering, by the one or more processors, a clinical intelligence data, specific to the patient location, from the internet, wherein the physiological index data further comprises the clinical intelligence data, the clinical intelligence data being selected from the group consisting of:
an environmental condition;
a seasonal ailment trend; and
an ailment outbreak.

17. The method of claim 11 further comprising the step of
transmitting the physiological index to the client device, wherein the client device provides a computerized interface between the one or more processors and an authorized personnel.

18. The method of claim 11 further comprising the step of generating a report, by the one or more processors, based on the sensor data and the physiological index data.

19. A non-transitory computer readable medium storing executable instructions which, when executed, cause one or more processors to perform the following steps for monitoring a patient, wherein the one or more processor is in communication with each other via a network, the one or more processor in communication with a storage unit, the steps comprising:
acquiring a sensor data from the patient by a plurality of sensors, the plurality of sensors operatively coupled to the patient, wherein each of the plurality of sensors is in communication with the one or more processors;
aggregating the sensor data from the plurality of sensors, by the one or more processors, wherein the sensor data is stored in the storage unit;
monitoring a behavior of the patient, using a client device in communication with the one or more processors, resulting from a response of the patient when an action is requested by the client device, wherein the behavior comprises a response time and a response accuracy, the response time indicating the time it takes for the patient to perform the requested action, and the response accuracy indicating the accuracy in which the patient performs the requested action;
collectively analyzing, by the one or more processors, a plurality of physiological index data and a correlation between the plurality of physiological index data;
comparing, by the one or more processors, the correlation to a medical protocol stored in the storage unit; and
generating, by the one or more processors, a physiological index based on the comparison, wherein the physiological index indicates diagnosis of the patient, the physiological index data comprising the sensor data acquired from at least one of the plurality of sensors, and the behavior.

20. The claim according to claim 19, further comprising the step of monitoring a movement of the patient, using a motion monitoring unit in communication with the client device, wherein the movement is requested by the client device as the action, the response time indicating the time it takes for the patient to perform the requested movement, and the response accuracy indicating the accuracy in which the patient performs the requested movement.
